(19) United States
(12) Patent Application Publication
(10) Pub. No.: US 2004/0143190 A1
(43) Pub. Date: Jul. 22, 2004

(54) Mapping neural and muscular electrical activity

(76) Inventor: Mark J. Schnitzer, Hoboken, NJ (US)

Correspondence Address: Docket Administrator (Room 3J-219), Lucent Technologies Inc., 101 Crawfords Corner Road, Holmdel, NJ, 07733-3030, US

(21) Appl. No.: 10/348,722

(22) Filed: Jan. 22, 2003

(51) Int. Cl. A61B 5/05

(52) U.S. Cl. 600/476

(57) ABSTRACT

A method produces an image that maps a level of electrical activity of electrically excitable membranes in a tissue mass The method includes positioning one end of an optical endoscope inside the tissue mass and illuminating a portion of the tissue mass with a light beam emitted from the endoscope. The method includes collecting light from the illuminated portion of the tissue mass to produce image data for one or more light intensity images and mapping the level of electrical activity of the electrically excitable membranes in the illuminated portion of the tissue mass based on the produced image data.

### BACKGROUND

1. Field of the Invention

The invention relates generally to medical diagnostic methods and systems.

2. Discussion of the Related Art

A variety of medical procedures produce maps of damaged areas of organs. Such maps are useful diagnostic tools for surgical procedures where one wants to remove damaged tissue without harming nearby undamaged tissue. The need for such maps is substantial in neural surgery where it is desirable that the least amount of normal neural tissue be damaged or removed.

Procedures for mapping damaged neural tissue rely on measurements of levels of neural activity. At organ surfaces, levels of neural activity have been determined from optical reflectivity measurements. The optical reflectivity of neural tissue changes in responsive to changes in the levels of neural activity therein.

While optical reflectivity measurements have enabled mapping neural activity at the surface of the brain, measurements of subsurface levels of neural activity are also of interest for surgical procedures deep in the brain. Unfortunately, optical absorption interferes with measuring optical reflectivities below the surface of the brain. For this reason, optical reflectivity is of limited usefulness in mapping damaged neural tissue deep in body organs.

### SUMMARY

Various embodiments provide methods for mapping activity of electrically excitable membranes found in neurons and muscle cells. In particular, the methods map activity levels deep in an animal or human tissue mass. The methods use invasive endoscopy to collect optical data indicative of such activity. From the optical data, the various methods produce an image or map of the level of such activity inside the tissue mass.

In one aspect, the invention features a method for mapping a level of electrical activity of electrically excitable membranes in a tissue mass. The method includes positioning one end of an optical endoscope inside the tissue mass and illuminating a portion of the tissue mass with a light beam emitted from the endoscope. The method includes collecting light from the illuminated portion of the tissue mass to produce image data for one or more light intensity images and mapping the level of electrical activity of the electrically excitable membranes in the illuminated portion of the tissue mass based on the produced image data.

In another aspect, the invention features a program storage medium encoding a computer executable program of instructions for performing the steps of a method. The steps include collecting light intensity data for first and second images of an interior portion of a tissue mass and producing an image of a level of electrical activity of excitable membranes in the portion of the tissue mass by comparing the light intensity data of the first and second images. The first image represents the interior portion in response to electrical or sensory stimulation. The second image represents the interior portion in the absence of the electrical or sensory stimulation.

In another aspect, the invention features a system for mapping electrical activity in electrically excitable membranes of a tissue mass. The system includes a light source, an optical endoscope coupled to receive light from the light source and to produce a light beam from the received light, a light detector, and a computer. The light detector is coupled to receive light that the endoscope collects from the tissue mass and to produce light image data from the received light. The computer is configured to store data for light intensity images of a portion of the tissue mass in response to receiving the light image data produced by the light detector. The computer is also configured to produce a map a level of electrical activity in electrically excitable membranes in the portion of the tissue mass based on the stored data.

### BRIEF DESCRIPTION OF THE DRAWINGS

FIG. 1A shows a setup that produces an image mapping electrical activity in electrically excitable membranes of a tissue mass based on reflected light images;

FIG. 1B shows a setup that produces an image mapping electrical activity in electrically excitable membrane activity of a tissue mass based on fluoresced light images;

FIG. 1C shows a setup that produces an image mapping electrical activity in electrically excitable membrane activity of a tissue mass based on light images produced by optical scanning;

FIG. 2A is a flow chart illustrating a method for mapping electrical activity in electrically excitable membranes of a tissue mass via reflected light imaging based on the setup of FIG. 1A;

FIG. 2B is a flow chart illustrating a method for mapping electrical activity in electrically excitable membranes of a tissue mass via fluorescent light imaging based on the setup of FIG. 1B;

FIG. 2C is a flow chart illustrating a method for mapping electrical activity in electrically excitable membranes of a tissue mass via scanning based on the setup of FIG. 1C; and

FIGS. 3A and 3B illustrate methods for optically scanning a tissue mass with a light beam made by a graded index (GRIN) optical fiber or GRIN lens.

In the various Figures, like reference numbers indicate elements with similar functions.

### DETAILED DESCRIPTION OF ILLUSTRATIVE EMBODIMENTS

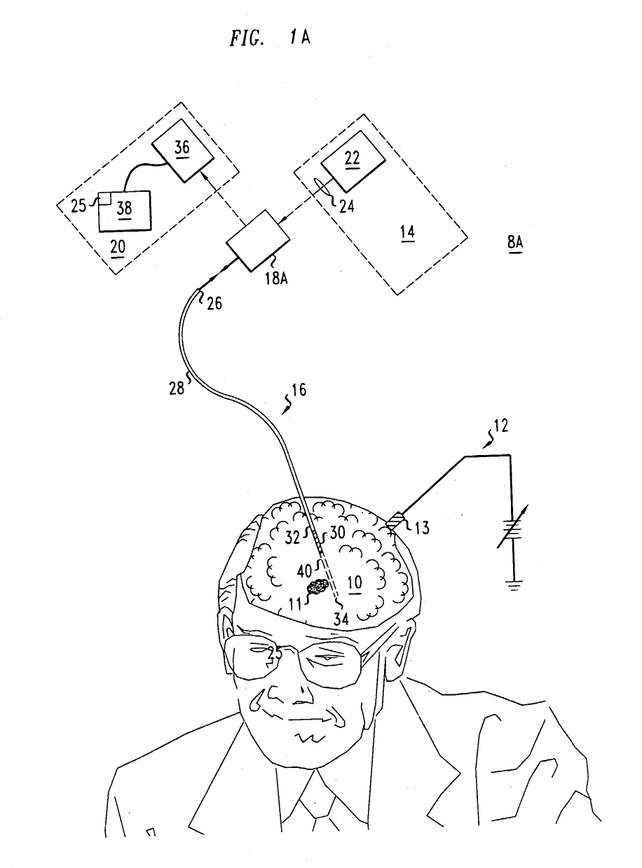

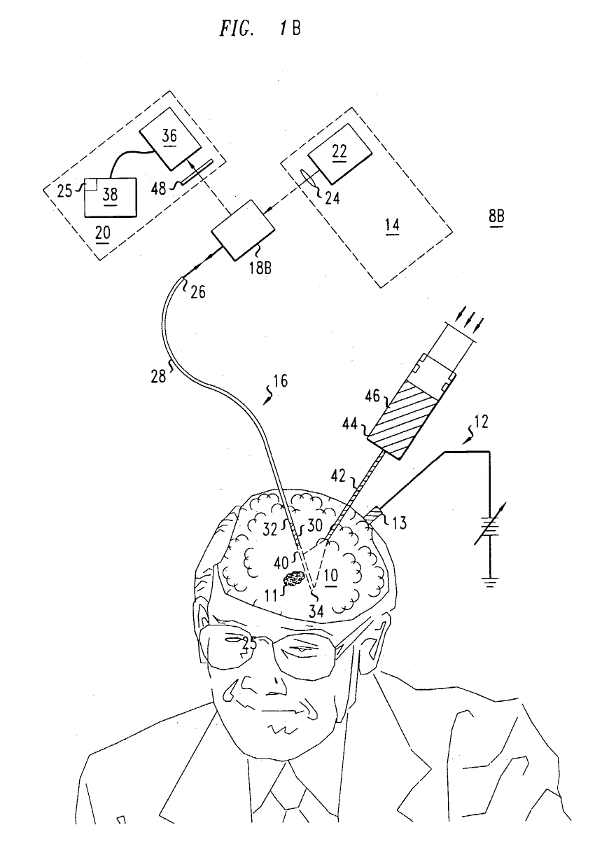

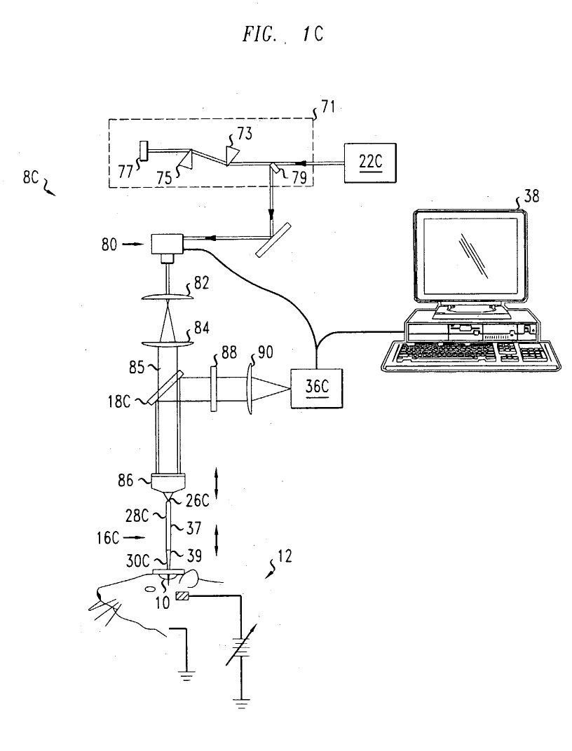

FIG. 1A shows a setup 8A for mapping levels of electrical activity in electrically excitable membranes that are located deep in a tissue mass 10. The electrically excitable membranes are located in neurons and muscle, i.e., smooth, striated, and cardiac. The electrical activity includes neural discharges and electrical changes at muscles membranes during muscular work.

The setup 8A uses invasive endoscopy to produce images in which light intensities are indicative of the electrical activity in various portions of the tissue mass 10. The setup 8A is able to map neural activity in deep tissue masses 10 such as the hippothalmus region of the brain. Maps of neural activity in tissues and organs are useful tools for finding a tumor 11 and for finding nerve activation centers for epilepsy.

The setup 8A includes a neural or muscular stimulator 12, an illumination system 14, an optical endoscope 16, an optical beam splitter 18A, and an optical imaging system 20. The neural or muscular stimulator 12 includes a voltage source and a probe 13 for generally stimulating electrical activity in the electrically excitable membranes of the tissue mass 10. The illumination system 14 includes a light source 22, e.g., a visible or near infrared laser, and collimation optics 24. The optical beam splitter 18A is partially reflective mirror or a birefringent prism that transmits light from the illumination system 14 to end 26 of the optical endoscope 16 and reflects light from the end 26 to the optical imagining system 20. The GRIN lens or fiber 16 produces a narrow light beam 34 for illuminating a local portion of the tissue mass 10.

The illustrated optical endoscope 16 is a GRIN lens or fiber that includes a relay GRIN fiber or lens 28 and an objective GRIN fiber or lens 30. The objective GRIN fiber or lens 30 is fused to a distal end 32 of the relay GRIN fiber or lens 28.

The compound GRIN fiber or lens produces an image with light reflected from the tissue mass 10.

Exemplary GRIN fibers or lens 16 include a simple GRIN lens with a length of between ½ to ¼ modulo a half-integer times the lens' pitch and preferably with a length of about ½ times the lens' pitch. Other exemplary GRIN fibers or lenses 16 16C also include compound GRIN lenses formed of a relay GRIN lens and an objective GRIN lens. The relay GRIN lens has a longer pitch than the objective GRIN lens. Exemplary objective and relay GRIN lenses have lengths equal to about ¼ and ¾ times their respective pitches.

Suitable GRIN fibers and GRIN lenses are described in U.S. patent application Ser. No. 10/082,870 ('870), filed Feb. 25, 2002; U.S. patent application Ser. No. 10/029,576 ('576), filed Dec. 21, 2001; and U.S. patent application Ser. No. 09/919,017 ('017), filed Jul. 30, 2001. The '870, '576, and '017 patent applications are incorporated herein by reference in their entirety.

The optical endoscope 16 also delivers light collected from a subsurface portion of the tissue mass 10 back to the optical beam splitter 18A. The optical beam splitter 18A directs a portion of this light to the optical imaging system 20. The optical imaging system 20 includes a light detector 36 that produces image data from the received light and a computer 38 that produces an image mapping the level of electrically excitable membrane activity from image data.

In some embodiments, the setup 8A enables mapping or imaging of electrical activity in electrically excitable membranes based on optical reflectance or optical fluorescent measurements.

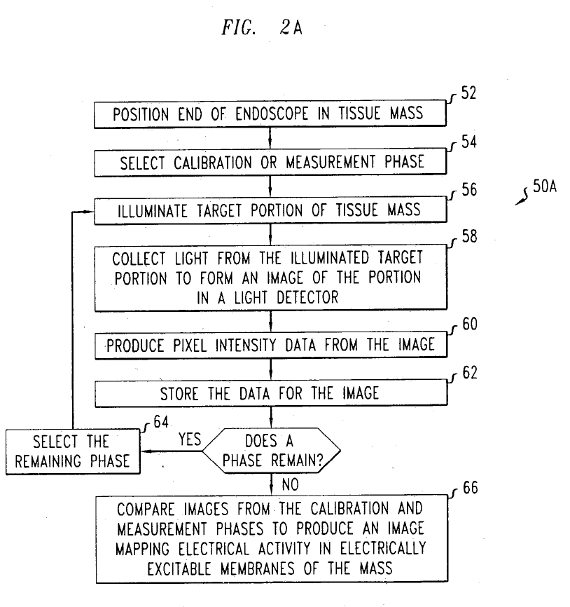

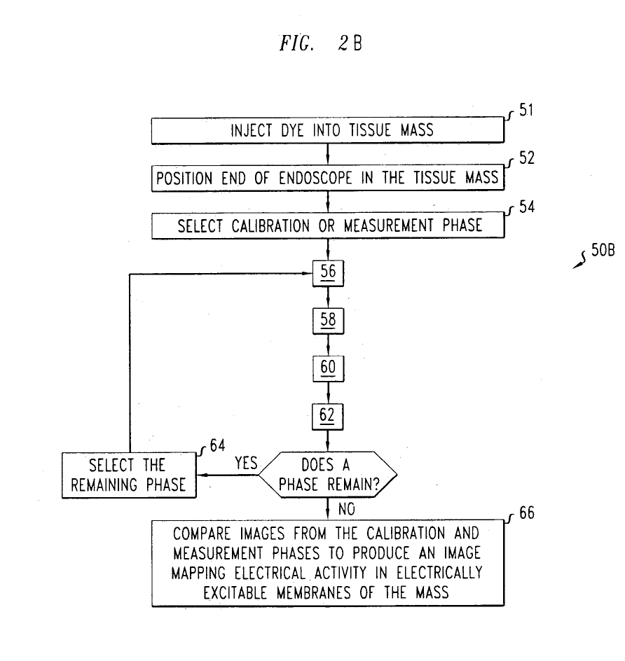

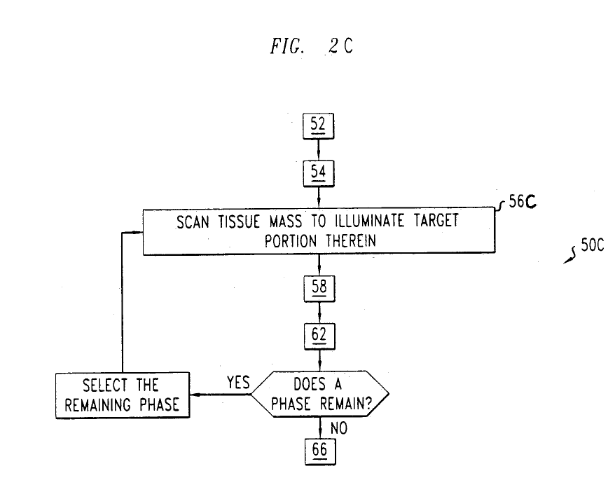

FIG. 2A illustrates a method 50A that uses optical reflectance measurements made with setup 8A of FIG. 1A to map the level of electrical activity in electrically excitable membranes of tissue mass 10. Prior to the measurements, distal end 40 of optical endoscope 16 is positioned inside the tissue mass 10 (step 52). The distal end 40 delivers a light narrow beam 34 that illuminates a region inside the tissue mass 10 where the activity will be mapped.

The method 50A includes selecting either a calibration phase or a measurement phase (step 54). During the measurement phase, electrical activity is generally stimulated in electrically excitable membranes of the neurons and/or muscles in the tissue mass 10. During the calibration phase, such electrical activity is not generally stimulated in the electrically excitable membranes of the tissue mass 12. Techniques for generally stimulating such electrical membrane activity include electrically stimulating the tissue mass 10 with voltage pulses of stimulator 12. The techniques for generally stimulating such electrical activity in neurons also include sensory stimulation. Exemplary types of sensory stimulation include: tone stimulation, light-flash stimulation, odor stimulation, taste stimulation, and touch stimulation. These types of sensory stimulation cause general stimulation of neural activity in neurons located in specific areas of the brain, e.g., auditory, visual, olfactory, taste, or touch sensory centers of the brain.

In the selected phase, the method 50A includes performing a sequence of steps to produce an image of a target portion of the tissue mass 10. The sequence includes illuminating the target portion of the tissue mass 10 with a collimated light beam 34 from the optical endoscope 16 (step 56). In response to the illuminating, a portion of the light emitted by the target portion is collected and delivered to the light detector 36 (step 58). The target portion of the tissue mass 10 reflects back a portion of the illumination light. The same optical endoscope 16 collects the reflected light and delivers a portion of this collected light to the light detector 36 via beam splitter 18A.

The delivered light produces a first reflection image of the target portion of the tissue mass 10 in light detector 36. The image indicates reflected light intensities in 1 or 2 dimensions transverse to the axis of the illuminating beam 34.

The sequence of steps also includes producing image data from the light intensities measured by the light detector 36 (step 60). The image data is a pixel-by-pixel map of the intensity of the reflected light received in the light detector 36. Finally, the data for this first image of the target portion of the tissue mass 10 is stored in a data storage device 25 of the computer 38 (step 62).

After performing the sequence of steps 56, 58, 60, and 62 in the selected calibration or measurement phase, the method 50A includes repeating the same sequence of steps in the remaining one of the measurement and calibration phases (64). The repeat of steps 56, 58, 60, and 62 produces data for a second reflected light image of the target portion of the tissue mass 10.

The method 50A also includes comparing the images from the calibration and measurement phases on a pixel-by-pixel basis to produce yet a third image that maps the level of electrical activity in the electrically excitable membranes in the targeted portion of the tissue mass 10 (step 66). The comparing step includes subtracting light intensities for pixels in the first image from light intensities for the same pixels in the second image.

The subtraction removes background reflected light intensities that are not associated with the target electrical membrane activity. In the case of neural activity, changes to a tissue's optical reflectance are typically small, e.g., less than about 1%. For this reason, such a background subtraction is typically needed to obtain optical reflectance intensities that are indicative of the absence or presence of neural activity. The pixel-by-pixel subtractions produce a third image in which intensity spots appear in portions of the tissue mass 10 with discharging neurons or electrically active muscle cells.

FIG. 2B illustrates an alternate method 50B, which uses optical fluorescence measurements to map the level of electrical activity in the electrically excitable membranes of tissue mass 10. The alternate method 50B uses an alternate setup 8B, which is shown in FIG. 1B.

The method 50B includes injecting dye into the tissue mass 10 prior to performing optical measurements (step 51). To inject the dye, a needle 42 of a syringe 44 introduces a solution 46 with the dye into the target portion of the tissue mass 10 as shown in FIG. 1B. The dye fluoresces in response to be illuminated with light of the wavelength produced by the light source 22. The amount of fluorescence by the dye molecules is responsive to the level of electrical activity in the electrically excitable membranes of the fluorescing portion of the tissue mass 10.

The dye is sensitive to a specific physiological change that is associated with electrical activity in membranes of neurons and/or muscle. Exemplary dyes are sensitive to ion concentrations or membrane voltages. These concentrations and voltages change during neural discharge and/or muscle cell contraction. The dye's sensitivity enhances the sensitivity of optical measurements to electrical activity in the electrically excitable membranes of neurons and/or muscles over sensitivities that are obtainable via reflectance measurements.

Exemplary dyes include lipophilic dyes, calcium-sensitive dyes, and sodium-sensitive dyes. The lipophilic dyes are absorbed by cell membranes and are sensitive to membrane changes produced during neural discharges and muscle contraction. The calcium-sensitive and sodium-sensitive dyes are sensitive to concentrations of calcium and sodium ions, respectively. The concentration of these ions changes during a neural discharge and/or a muscle contraction.

Exemplary ion-concentration sensitive and membrane-voltage sensitive dyes are available from Molecular Probes Company of 29851 Willow Creek Rd., Eugene Oreg. 97402. The ion concentration sensitive dyes include Ca²⁺ sensitive dyes that Molecular Probes sells under the product names: Calcium-Green 2, Fluo-5, and Indo-1. The voltage sensitive dyes include dyes that Molecular Probes sells under the product names: JC-9, di-8-ANEPPS, and di-4-ANEPPS.

The method 50B also includes performing steps 52, 54, 56, 58, 60, 62, and 64, which were already described with respect to method 50A of FIG. 2A. In steps 58 and 60, fluoresced light rather than reflected light produces the images of the targeted portion of the tissue mass 10. The fluoresced light has a different wavelength than illumination light from the light source 22.

To produce an image from fluoresced light, the optical beam splitter 18B includes a dichroic slab. The dichroic slab separates fluoresced light and illumination light based on wavelength. Some embodiments of the setup 8B also have a filter 48 that removes residual light at the illumination wavelength from the beam directed to the light detector 36.

In an alternate method, an optically opaque dye replaces the fluorescent dye in the method 50B. The absorbance of the opaque dye is responsive to the level electrical activity in the electrically excitable membranes of the tissue mass 10. In such embodiments, reflected light images are again used to make an image mapping such activity in the tissue mass 10.

Another alternate method 50C uses scanned images, which are made with setup 8C of FIG. 1C, to map the level of electrical activity in electrically excitable membranes of a tissue mass 10. The scanned images are produced by fluorescence, which is produced by multi-photon absorption events in the tissue mass 10. The absorption events either occur in biological molecules of the tissue mass itself or in dye molecules that have been injected into the tissue mass 10. Such multi-photon absorption events need strong light intensities. For that reason, fluorescence rates are only significant in the intensely illuminated portions of the tissue mass 10, e.g., the focused waist of the illumination beam. For that reason, the method 50C produces images with a higher resolution than those formed by the methods 50A and 50B.

Referring to FIG. 1C, the setup 8C includes a pulsed laser 22C that provides the high intensity optical pulses needed to generate two-photon absorption events. Exemplary pulsed lasers 22C include ultra-fast pulsed Ti-sapphire lasers that produce femto-second or pico-second pulse lengths. The pulsed laser 22C sends the optical pulses to a compensator 71 that pre-compensates for chromatic dispersion, which could otherwise lower pulse intensities. The compensator 71 sends the pre-compensated optical pulses to an optical delivery system, which transmits the pulses to an optical endoscope 16C. The optical endoscope 16C delivers the high intensity optical pulses to the target portion of the tissue mass 10.

The compensator 71 includes a pair of Brewster angle prisms 73, 75, a reflector 77, and a pick off mirror 79. The compensator 71 functions as a double-pass device, in which light passes through each prism 73, 75 twice. The pick-off mirror 79 deflects a portion of the beam of pre-compensated pulses from the compensator 73 and sends the deflected portion of the beam to the optical delivery system.

The optical delivery system includes a pair of x-direction and y-direction beam deflectors 80, a telescopic pair of lenses 82, 84, a dichroic mirror 18C, and an insertion lens 86.

Exemplary x, y-direction beam deflectors 80 include galvanometer-controlled mirrors, acousto-optic deflectors, and electro-optic deflectors. The x-direction and y-direction beam deflectors 80 steer the beam in lateral directions thereby producing a two-dimensional scan of a lateral portion of the tissue mass 10. The computer 38 controls the x-direction and y-direction beam deflections that are generated by beam deflectors 80. Thus, the computer 38 controls scanning of the tissue mass 10 in directions lateral to the beam direction.

From beam deflectors 80, optical pulses pass through a telescopic pair of lenses 82, 84. The lenses 82, 84 expand the beam diameter to produce an expanded illumination beam 85. The expanded beam 85 passes through dichroic mirror 18C and is transmitted to insertion lens 86, i.e., a high numerical aperture lens. The diameter of the expanded beam 85 matches the entrance pupil of the insertion lens 86. The insertion lens 86 focuses the expanded illumination beam 85 to a spot on or near the external end face of the GRIN endoscope 16C, i.e., a spot located in the interior of the tissue mass 10.

The imaging system 8C has a dual focus mechanism (not shown) that enables independently adjusting the distance of the end face of the optical endoscope 16C below the surface of the tissue mass 10 and the distance between the insertion lens 86 and the optical endoscope 16C. The dual focusing mechanism enables fine adjustments of the depth of the optical endoscope's focal plane in the tissue mass 10 without requiring movements of the optical endoscope 16C itself.

Portions of the tissue mass 10 fluoresce light in response to two-photon absorption events. Part of the fluoresced light is collected by the optical endoscope 16C, which delivers the collected light to insertion lens 86. From the insertion lens 86, dichroic mirror 18C deflects the collected light to a chromatic filter 88. The chromatic filter 88 removes wavelengths outside the fluorescence spectrum and delivers the remaining light to a focusing lens 90. The focusing lens 90 focuses the remaining light onto a photo-intensity detector 36C, e.g., a photomultiplier or avalanche photodiode. The photo-intensity detector 36C produces an electrical signal indicative of the total intensity of the received fluorescence light and transmits the electrical signal to computer 38, i.e., an electronic processor and controller. The computer 38 uses intensity data from the photo-intensity detector 36C and data on the x- and y-deflections of the illuminating beam 85 to produce a scan image of a target portion of the tissue mass 10.

The optical endoscope 16C is either a GRIN lens or a GRIN fiber that forms a focused scanning spot inside the tissue mass 10. Exemplary optical endoscopes 16C include a simple GRIN lens with a length of less than ¼ pitch modulo a half-integer times the lens' pitch and preferably with a length of about ½ times the lens' pitch. Exemplary optical endoscopes 16C also include compound GRIN lenses formed of a relay GRIN lens and an objective GRIN lens. The relay GRIN lens has a longer pitch than the objective GRIN lens. Exemplary objective and relay GRIN lenses have lengths of less than ¼ pitch modulo a half-integer times their respective pitches.

In the various embodiments, the numerical aperture of optical endoscope 16C is large enough to accept the entire cone of light incident on its external end face 26C Thus, light for exciting multi-photon processes is not lost at the external end face 26C.

Suitable GRIN lenses and fibers are described in the '870 and '576 patent applications incorporated herein.

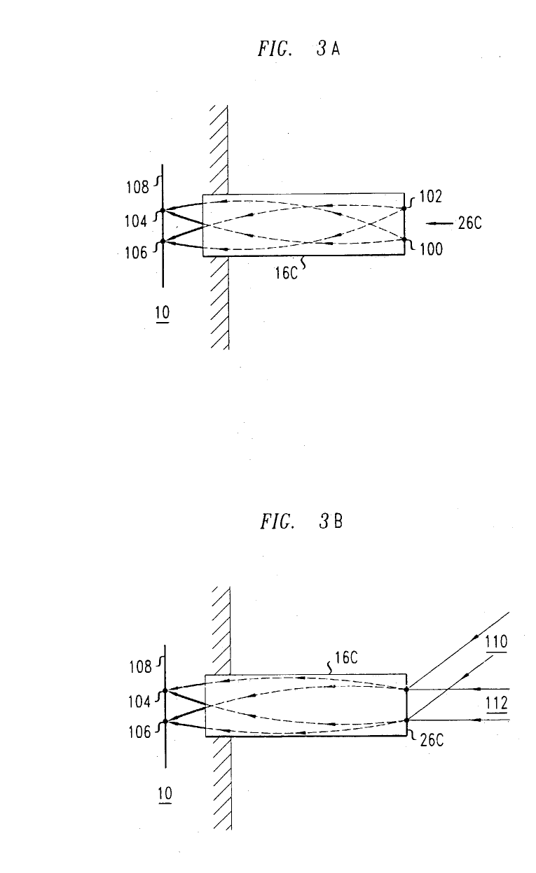

FIGS. 3A and 3B illustrate methods for optical scanning a portion of the tissue mass 10 with a light spot from the GRIN optical endoscope 16 of FIG. 2C.

In FIG. 3A, a focused light beam scans the external end face 26C of the optical endoscope 16C. From each spot of light 100, 102 on the external end face 26C, the GRIN optical endoscope 16C produces a second focused spot of light 104, 106 in a focal plane 108 located in the tissue mass 10. Thus, optically scanning the external end face 26 of the optical endoscope 16C produces an optical scan of a portion of the plane 108.

In FIG. 3B, a collimated light beam 110, 112 is pivoted to change its incidence angle on the external end face 26C of the optical endoscope 16C. Pivoting the incidence angle between the directions of the collimated light beams 110, 112 causes a focused light spot to scan the tissue mass 10 from point 104 to point 106 on the focal plane 108 located therein.

FIG. 2C illustrates the method 50C, which maps the level of electrical activity in electrically excitable membranes of the tissue mass 10 with the setup 8C of FIG. 1C.

The method 50C includes performing steps 52 and 54 as already described in method 50A.

The method 50C also includes optically scanning the tissue mass 10 to illuminate a target portion therein (step 56C). To perform the scan, the laser 22C produces pulses, which are transmitted to the external end face 26C of the endoscope 16C to perform the scan. The x, y beam deflectors 80 produce a 1-dimensional or 2-dimensional raster scan of the incidence angle or the incidence position of the beam of laser light pulses on the external end face 26C. Scanning the incident laser light beam causes a focused light spot to scan a spatial target portion inside the tissue mass 10.

The method 50C also includes performing steps 58, 60, 62, 64, and 66 as already described in method 50A. During image forming step 58, an image of the scan spots in the light detector 36C. The light detector 36C measures the received portion of the total intensity of fluoresced or harmonic light, which is produced by two-photon events or nonlinear optical processes in the scanned spots of the tissue mass 10. During step 60, the computer 38 uses the measured intensities of fluoresced or harmonic light from the light detector 36C and calculated positions of optical scan spots to construct an image for one pixel of the tissue mass 10. As the scan continues the computer 38 produces an image of the target portion of the tissue mass in a sequential pixel-by-pixel manner. During step 66, the programmed computer 38 compares corresponding pixels in the scan images from the calibration and measurement phases to produce an image. The image maps the level of electrical activity in the electrically excitable membranes in neurons and/or muscle of the target portion of the tissue mass 10.

Referring to FIGS. 2A-2C, some embodiments of methods 50A, 50B, and 50C use computer 38 to perform or control one or more of steps 54, 58, 56, 60, 62, 64, and 66. The computer 38 executes an executable program of instructions. The program is stored in computer executable form in a data storage medium, e.g., the data storage device 25 of FIGS. 2A-2B. Exemplary data storage media include optical disks, magnetic tapes, magnetic disks, read-only memories, and active memories.

In these embodiments, the computer 38 also performs general electrical stimulation of electrical activity in electrically excitable membranes of neurons and/or muscle cells by operating the neural or muscle stimulator 12. Thus, the computer 38 uses voltages to stimulate such electrical membrane activity during or prior to collection of reflected, fluoresced, or harmonic light in steps 56 and/or 58 of the measurement phase.

In alternate embodiments of methods 50A-50C of FIGS. 2A-2C, the optical response to electrical activity in electrically excitable membranes of neuron or muscle cells is intense. For that reason, a calibration phase is not needed. Then, the images that map levels of electrical activity in electrically excitable membranes are made directly from images produced during general electrical stimulation, i.e., images of the measurement phase. In these embodiments, the maps do not involve subtraction of background light intensities from calibration phase measurements.

From the disclosure, drawings, and claims, other embodiments of the invention will be apparent to those skilled in the art.

What is claimed is:

1. A method, comprising:
positioning one end of an optical endoscope inside a tissue mass;
illuminating a portion of the tissue mass with a light beam emitted from the endoscope;
collecting light from the illuminated portion of the tissue mass to produce image data for one or more light intensity images of the illuminated portion of the tissue mass; and
mapping a level of electrical activity of electrically excitable membranes in the illuminated portion of the tissue mass based on the image data.

2. The method of claim 1, further comprising:
stimulating the activity in the tissue mass with an electrical stimulator; and
wherein said collecting produces image data for an image of the tissue mass when electrically stimulated by the stimulator.

3. The method of claim 1, wherein said collecting produces image data for an image of the tissue mass when not electrically stimulated by the stimulator.

4. The method of claim 1, further comprising:
injecting a dye into the tissue mass prior to performing the collecting, the dye being optically responsive to the level of the electrical activity.

5. The method of claim 4, wherein a fluorescence rate of the dye is responsive to a level of neural discharge activity.

6. The method of claim 4, wherein the dye is one of sensitive to a calcium ion concentration, sensitive to a sodium ion concentration, sensitive to a membrane voltage, and lipophilic.

7. The method of claim 1, wherein the positioning comprises placing the one end of the endoscope inside brain tissue of a man or animal.

8. The method of claim 1, wherein the illuminating includes optically scanning the portion of the tissue mass with the light beam from the endoscope.

9. The method of claim 8, wherein the illuminating produces one of multi-photon absorption events and harmonic light in the tissue mass; and wherein said one or more light intensity images are formed from one of fluorescence light emitted in response to said multi-photon absorption events and the harmonic light.

10. A program storage medium encoding a computer executable program of instructions for performing a sequence of steps of a method, the steps comprising:
collecting light intensity data for first and second images of an interior portion of a tissue mass, the first image representing the interior portion in response to electrical or sensory stimulation, the second image representing the interior portion in absence of said electrical or sensory stimulation; and
producing an image of a level of electrical activity in electrically excitable membranes of the portion of the tissue mass by comparing the intensity data of the first and second images.

11. The medium of claim 10, wherein the collecting step comprises scanning a light beam through the interior portion of the tissue mass.

12. The medium of claim 11, wherein the collecting step further comprises producing said data from measured intensities of light fluoresced from the interior portion.

13. The medium of claim 11, wherein the collecting step further comprises:
electrically stimulating the portion of the tissue mass to produce general neural activity therein during the collecting of the light intensity data for the first image.

14. The medium of claim 11, wherein the producing includes comparing the intensity data for pixels in the first image to corresponding pixels in the second image.

15. A system for mapping electrical activity in electrically excitable membranes of a tissue mass, comprising:
a light source;
an optical endoscope coupled to receive light from the light source and to produce a light beam from the received light;
a light detector coupled to receive light collected from the tissue mass by the endoscope and to produce light image data from the received light; and
a computer configured to store data representative of light intensity images of a portion of the tissue mass in response to receiving the light image data produced by the light detector and configured to produce a map of a level of electrical activity in the electrically excitable membranes in the portion of the tissue mass based on the stored data.

16. The system of claim 15, further comprising:
a neural stimulator capable of electrically stimulating the tissue mass; and
wherein the processor is configured to map neural activity by comparing data for light intensity images produced in the absence and presence of said electrically stimulating.

17. The system of claim 15, wherein the light detector is configured to measure intensities of light produced by multi-photon absorption events or harmonic light in the tissue mass.

18. The system of claim 17, wherein the endoscope is selected from the group consisting of a compound GRIN lens and a compound GRIN fiber.
